(19) United States
(12) Patent Application Publication
(10) Pub. No.: US 2006/0099281 A1
(43) Pub. Date: May 11, 2006

(54) Herbal blood-pressure lowering and blood-lipid lower food

(76) Inventor: Yingbin Quan, Beijing (CN)

Correspondence Address: JACOBSON HOLMAN PLLC, 400 SEVENTH STREET N.W., SUITE 600, WASHINGTON, DC, 20004, US

(21) Appl. No.: 10/548,123

(22) PCT Filed: Apr. 29, 2003
(86) PCT No.: PCT/CN03/00318

§ 371 Date: Dec. 1, 2005

(30) Foreign Application Priority Data
Mar. 3, 2003 (CN) 03105015.8

(51) Int. Cl. A61K 36/482

(52) U.S. Cl. 424/757

(57) ABSTRACT

The invention provides a kind of food having blood-pressure lowering and blood-lipid lowering properties, the food include cold drink, beverage, flour and cake. The cold drink is obtained by boiling granular semen cassiae together with water in 1:5 proportion for 15 minutes, continuing heating under mild temperature for 30 minutes, filtering, adding liquid containig semen cassiae ingredients to the raw materials of cold drink. The cake is obtained by corperating semen cassiae residues into the flours. The flour is obtained by milling the semen cassiae into fine powder, corporating the powder into the flour.

### TECHNICAL FIELD

The invention involves a kind of food that mixes with Chinese herbal medicine, and that belongs to the technical field of medicable food, which refers to a kind of Blood-pressure lowering and blood-lipid lowering foods.

### TECHNICAL BACKGROUND

Since Li Shizhen, the outstanding pharmacological expert of the Ming Dynasty, undertook initial try in the past, Chinese have begun to fully exploit the mysteries of the Chinese herbal medicine, which contribute to human brilliantly. However, since the human came into being, and among those more than 80 billion ancestors who died before us, was it only several billions which died of the hypertension and the high fat? The cardiovascular and cerebrovascular diseases even become the deadly killer to the human in the present age! At least within three decades before the human can draw the illustration of genetic-gene, it is really an emergency for finding the “unbeatable rival” in the Chinese herbal medicines to resist above those killers which take our kindred away every now and then! But at present there are no simple and effective medicable foods except the specific traditional Chinese herbal medicines. Especially, the kind of Blood-pressure lowering and blood-lipid lowering food that can prevent above diseases and that is made by only incorporate a kind of ingredient of Chinese traditional medicine that is easy to obtain into the foods.

### OPEN OF THE INVENTION

For the problems above, according to the before-mentioned ideas, the purpose of this invention is to provide a kind of “Blood-pressure lowering” and “blood-lipid lowering” and “loss weight” herbal food by incorporating “Cassia tora L” into the daily diet.

The technological plan adopted by this invention is as follows:

The Herbal Blood-pressure lowering and blood-lipid lowering foods include four classes such as the cold drinks, beverages, flour, and cakes that have the function of lowering the blood fat, the characteristics are: incorporate Herbal Cassia tora L powder into above four kinds of foods according to different proportioning, and then make by common craft to gain above four kinds of Herbal Blood-pressure lowering and blood-lipid lowering foods.

Methods in Detail:

1. Cold Drinks:

Confect Herbal Cassia tora L grains and water by 1 kg and 5 kg weight ratio, boil for 15 minutes, and then go on for 30 minutes by slow fire, namely cook for 45 minutes together; then filtrate concurrent liquid and solid to leach out the Herbal Cassia tora L residue to stand-by, and reserve the liquid that contains ingredients of Herbal Cassia tora L to join into the material that is ready to make the cold drinks. Dry and grind above the Herbal Cassia tora L residue filtrated into powder.

2. Cake Type

The abovementioned solid residues are dried and grinded into pieces to become the dry semen cassiae powders: Those powders will be mixed into flour with a ratio that is varied for different persons. The people whose families see the disease histories of hypertension and hyperlipemia need a bigger dosage, so the weight ratio of semen cassiae residuals to flour should be from 6-60%. Other people whose families have no disease history are common persons taking cassiae for health care, so that weight ratio should be from 0.01-1.99%. In accordance with a general cake-making technology, the flour will be made into a snack food for curing hypertension and hyperlipemia.

3. Flour Type

The semen cassiae residuals are grinded into white-flour-style fine powders. Those powders will be mixed in flour that will be used for curing hypertension and hyperlipemia with a ratio as follows: Those residuals will be mixed into flour with a ratio that is varied for different persons. The people whose families see the disease histories of hypertension and hyperlipemia need a bigger dosage, so the weight ratio of semen cassiae residuals to flour should be from 5-20%. Other people whose families have no disease history are common persons taking cassiae for health care, so that weight ratio should be from 0.01-4.99%. The mixture will be churned up evenly in a big machine for making a variety of flour food.

### BEST WAYS FOR REALIZING THE INVENTION

### EXAMPLE 1

Add the 0.5 kg semen cassiae powders into the 9.5 kg flour. Mix them evenly in the big machine for making flour food such as dough.

### EXAMPLE 2

Dry and grind the 100 g-more semen cassiae residuals into flour-style powders and then mix them into the 1900 g flour. Use the flour to make snack food in accordance with a general technology.

### EXAMPLE 3

Add the 2 kg semen cassiae into the 10 kg water and boil it for 15 minutes. Then, further boil the cassiae on the middle level of fire for 30 minutes, until it has been cooked for 45 minutes totally. Use the boiled liquid to mix the milk and sugar juices whose weights are same as this liquid. Then, add the mixed liquid into the raw materials of beverage, in order to make the cold drinks for curing hypertension and hyperlipemia.

### EXAMPLE 4

Add the 10 g semen cassiae powders into the 100 kg flours and mix them evenly. Then, make them into snack food in accordance with a general technology. This food can be provided on the prevention and health-care purposes for the people whose families see no disease history.

### EXAMPLE 5

Add the 60 g semen cassiae powders into the 240 g flours and mix them evenly. Then, make them into snack food in accordance with a general technology. This food can be provided on for the people whose families see the disease histories of hypertension and hyperlipemia.

### EXAMPLE 6

Add the 60 g semen cassiae powders into the 3940 kg flours and mix them evenly. Then, make them into snack food in accordance with a general technology. This food can be provided on the prevention and health-care purposes for the people whose families see no history of hypertension and hyperlipemia.

### INDUSTRIAL APPLICABILITY

The invention has undergone researches and experiments for a long period. According to the long-term researches and surveys, the directions for making the food with semen cassiae not only prove effective on preventing and curing the hypertension and hyperlipemia, but also are usable for treating with weight loss. However, the food is unfavorable to hypotensives and the persons who often suffer from diarrhea. Cold drinks, cakes and flour food that are made with the directions are accepted easily in customers. With the unperceptive adjustment of food and drink, diseases which do great harms to human health become curable.

The semen cassiae has been recorded completely for many times in historical pharmacopoeias. The Chinese name “Jue Ming Zi” was originated from a book “Shennong's Pharmacopoeia”. Also it was called “Cao Jue Ming”, “Yang Ming” (in “Wupu's Pharmacopoeia”), “Yang Jiao” (in “Guang Ya”), “Mati Jueming” (by Tao Hongjing), “Huan Tong Zi” (in “Official Medical History”), “Gou Shi Dou” (in “Abstracts of Wild Herbs' Pharmic Attributes”), “False Mung Bean” (in “Records of Chinese Officinal Plants”), “Ma Ti Zi” (in “Records of Jiangsu's Officinal Plants”), “Qian Li Guang” (in “Records of Shanxi's Officinal Plants”), “Qin Jue” (in “Records of Shaanxi's Officinal Plants”), “Yang Jiao Dou” (in “Records of Guangdong's Officinal Plants”), “Wild String Bean” (in “Records of Jiangxi's Officinal Plants”), “Zhu Gu Ming”, “Zhu Shi Lan Dou”, “Xi Ye Zhu Shi Dou”, “Ye La Zi”, “Yang Wei Dou” and “Tai Ga Duo Jie” in Tibetan, etc.

The semen cassiae is the mature seed of leguminous plant “cassiae.” The cassiae is named (in “Classic of Materia”) as: “Ye Guan Men”, “Yang Chu Zu”, “False Yang Jiao Rong”, “False Peanut”, “Ye He Cao” and “Wild Peanut”. It is the herbaceous plant of one-year vegetal term with one meter height, vertical stalk, many upper branches and all-body short tender hair. The leaves grow with each other and look like double feathers. The leafstalk sees a ditch; the leaf axis features a gland between two small leaves; the supporting leaves are linear in shape and have a caducity; and there are three pairs of small leaves which look like inverted eggs with the 2-3 cm length. In addition, the leave ends are round in shape with slight sharps; the bottoms are cuneal or almost round with one inclined side; the leaves are integrifolious; the upper ones are nearly without hair; and the lower ones are covered with tender hair. The pairs of flowers grow on the axilla with the total-one-centimeter footstalk and tender hair. There are 5 calyxes with egg shape and outside tender hair. There are 5 petals which look like inverted eggs with the short claws and yellow color. And there are 10 stamens; 3 upper ones are degraded; and 7 lower ones are completely developed. The germen is thin, long, curved and haired with a footstalk, an extremely short column and a head-like chapiter. The legume fruits are linear, slightly flat and arched curve in shape, with the 15-14 cm length, the 46 mm diameter and the combed tender hair. Most of the seeds are rhombic with the grey-green color and luster. The florescence is from 6-8 months and the fruiting term is from 9-10 ones. The plant grows on hills and riverbanks wildly or is humanly cultivated in most of regions in China. The whole body or the leaves (wild peanuts) of the plant feature the officinal values.

The pharmacology and purpose of semen cassiae are as follows:

1. The semen cassiae is used to cure blind people who have no disease but cannot see anything clearly. The direction is: Have the filtered 2-liter semen cassiae with porridge, and the taboo diets include fish, garlic, pork and hot dishes. (according to “Monks' Collected Directions”)
2. The semen cassiae is used to cure red-skin galls with the direction: Fry and grind the semen cassiae and mix it with tea. Then place the mixture on the patient's two temples. Replace the mixture when it is dried. Also this can cure intermittent headache. (according to “Zhai Yuan Directions”)
3. The semen cassiae is used to cure moon blindness with the direction: Make a powered medicine with two liangs of semen cassiae and one liang of fructus cochiae. Drink porridge with the medicine of one qian on weight after have a meal (according to “Sheng Hui Directions”).
4. The semen cassiae is used as a medicinal herb to liver and eye (according to “Sheng Hui Directions”)
5. The semen cassiae is used as a medicinal herb to acute colonitis (according to “Chinese Traditional Medicine Manual in Hebei Province”)
6. The semen cassiae is used as a medicinal herb to hypertension (according to “Herbs in Jiangxi Province”)
7. The semen cassiae is used as a medicinal herb to infantile malnutrition (according to “Herbs in Jiangxi Province”)
8. The semen cassiae is used as a medicinal herb to tinea (according to “Su Shenliang's Directions”)
9. The semen cassiae is used as an antibacterial: The alcohol extracts from semen cassiae seeds are able to ban MRSA, C. diphtheriae and the bacteria of typhoid, paratyphoid and colitis; and the water extracts have no effect. Water medicament: In a test tube, the (1:4) medicament is able to ban some dermal epiphytes for different degrees. The little chrysophanol the semen cassiae contains may have some relationship with the slight antibacterial and evacuant purposes.
10. The semen cassiae is able to lower serum cholesterol and cure hyperlipemia.
11. Other purposes: Relive fever, brighten eyes, moisten intestines and defecate; also the semen cassiae is used for dermal epiphytes, indigestion, worm, parasite, calmness and restoration.

1. A kind of Herbal Blood-pressure lowering and blood-lipid lowering food, including four classes such as the cold drinks, beverages, flour, and cakes that have the function of lowering the blood fat, the characteristics rest with: incorporate Herbal Cassia tora L powder into above four kinds of foods according to different proportioning, and then make by common craft to gain, above four kinds of Herbal Blood-pressure lowering and blood-lipid lowering foods.

2. Herbal Blood-pressure lowering and blood-lipid lowering foods stated in the Demand 1, the characteristics rest with: the method that make the cold drinks is:
Confect Herbal Cassia tora L grains and water by 1:5 weight ratio, boll for 15 minutes, and then go on for 30 minutes by slow fire, namely cook for 45 minutes together; then filtrate concurrent liquid and solid to leach out the Herbal Cassia tora L residue to stand-by, and reserve the liquid that contains ingredients of Herbal Cassia tora L to join into the material that is ready to make the cold drinks, that is Herbal Blood-pressure lowering and blood-lipid lowering cold drinks made.

3. Herbal Blood-pressure lowering and blood-lipid lowering foods stated in the Demand 1, the characteristics rest with: the method that make the cakes is:
Dry and grind above the Herbal Cassia tora L residue filtrated into powder, and join it into the flour, the incorporate proportion depends on the needs of different crowd: the crowd which has the medical history of hypertension and high blood fat needs bigger amount. The powder of the residue of the Herbal Cassia tora L should account for 6-60% in the flour; for the normal crowd which haven't the family medical history and who take the health care as the main purpose, the powder of the residue of the Herbal Cassia tora L should account for 0.01-1.99% in the flour, which make the Blood-pressure lowering and blood-lipid lowering cakes with common technological process.

4. Herbal Blood-pressure lowering and blood-lipid lowering foods stated in the Demand 1, the characteristics rest with: the method that make the flour is:
Grind the grain of the Herbal Cassia tora L filtrated into powder like flour, and join it into the flour to make Blood-pressure lowering and blood-lipid lowering flour, the concrete proportion is: for the crowd which has the medical history of hypertension and high blood fat, the powder of the Herbal Cassia tora L accounts for 5-20% in the flour; for the normal crowd which haven't the family medical history and who take the health care as the main purpose, the powder of the Herbal Cassia tora L accounts for 0.01-4.99% in the flour; homogenize the admixture of the powder of the Herbal Cassia tora L and the flour with big machine after incorporating, then to make kinds of foods.
